(19) United States
(12) Patent Application Publication
(10) Pub. No.: US 2026/0115095 A1
(43) Pub. Date: Apr. 30, 2026

(54) DETERMINATION OF CARDIAC COMPRESSION LOCATION FROM ELECTROCARDIOGRAM SIGNALS

(71) Applicant: IMPACK-CPR INC., Montréal (CA)

(72) Inventors: Lawrence Leroux, Montréal (CA); Louis-Philippe Fortier, Montréal (CA); Étienne Fortin, Longueil (CA); Richard LaBerge, Montréal (CA); François LaBerge, Montréal (CA)

(21) Appl. No.: 18/972,066

(22) Filed: Dec. 6, 2024

Related U.S. Application Data
(60) Provisional application No. 63/606,733, filed on Dec. 6, 2023.

(51) Int. Cl. A61H 31/00; A61B 5/00; A61B 5/257; A61B 5/346; A61N 1/39

(52) U.S. Cl. CPC A61H 31/006; A61B 5/257; A61B 5/346; A61B 5/6823; A61B 5/6833; A61B 5/7415; A61B 5/742; A61H 31/005; A61N 1/3904; A61N 1/3993; A61B 2560/0468; A61B 2562/046; A61H 2201/1207; A61H 2205/084; A61H 2230/04

(57) ABSTRACT

A pad carrying an array of ECG electrodes can be used to measure ECG signal amplitudes and AMSA, a signal analyzer determines a cardiac position with respect to the pad, and an indication of cardiac position with respect to indicia can be provided on a top surface of the pad for output to the user to guide in performing chest compressions during CPR. The pad may integrate a defibrillator electrode. The apparatus may be an automated external defibrillator (AED).

This patent application claims priority to U.S. provisional patent application 63/606,733 filed Dec. 6, 2023 and titled “Autonomous Cardiopulmonary Resuscitation Device”, the contents of which are hereby incorporated by reference in their entirety.

### TECHNICAL FIELD

This patent application relates to tools used for cardiopulmonary resuscitation (CPR) and tools used to analyze electrocardiogram (ECG) signals.

### BACKGROUND

Despite recent technological advances, rates of survival of a patient suffering from cardiac arrest to hospital discharge are still under 10% in most regions of the world. Chest compressions, in cardiac arrest, are currently performed either manually or automatically by mechanical chest compression devices on the lower half of sternum at a specified rate and depth in accordance with American Heart Association (AHA) guidelines. However, patients may have different chest dimension, chest compliance, cardiac position, cardiac arrest etiology, and myocardial relaxation capacity. These characteristics are not currently accounted for during resuscitation. Recent literature has shown that optimal chest compression characteristics (site, rate, duty cycle, depth of compression and decompression) may be different from patient to patient. It is known that the position of chest compressions with respect to the patient's cardiac position can play a significant role in the survival rate of the patient, namely survival is essentially zero if the position of cardiac compressions during CPR is merely a few centimetres away from the center of the left ventricle (Resuscitation, Volume 138, P8-14, May 2019, & Resuscitation, Volume 84, P1203-1207, September 2013).

CPR may be administered with measurement of a physiological parameter of the patient providing feedback as to whether chest compressions as currently administered are being effective. However, pre-CPR guidance specific to selecting a chest compression location corresponding to cardiac position for use during CPR is not available.

### SUMMARY

In some embodiments, there is provided an apparatus having a pad carrying an array of ECG electrodes is used to measure ECG signal amplitudes and AMSA, a calculator calculates a cardiac position with respect to the pad, and an indication of cardiac position with respect to indicia provided on a top surface of the pad is output to the user to guide in performing chest compressions during CPR. The pad may integrate a defibrillator electrode. The apparatus may be an automated external defibrillator (AED). One or more electrodes may be added as reference.

In some embodiments, the pad may be used in patients with sinus rhythm who are at high risk of cardiac arrest. In such cases, the pad may inform the clinician of the optimal chest compression site to use in the event of a cardiac arrest. A temporary or permanent mark may be left on the skin to identify this site.

In some embodiments, there is provided an automated CPR apparatus having a mechanical chest compression device, a pad carrying an array of ECG electrodes is used to measure ECG signal amplitudes, a calculator calculates a cardiac position with respect to the pad, and at least one of: an indication of cardiac position with respect to indicia provided on a top surface of the pad is output to the user to position the mechanical chest compression device during CPR; and a positioning actuator is responsive to the calculated cardiac position to position the mechanical chest compression device during CPR.

### BRIEF DESCRIPTION OF THE DRAWINGS

The invention will be better understood by way of the following detailed description of embodiments of the invention with reference to the appended drawings, in which:

FIG. 1 is an illustration of a human chest showing the ribcage and the position of the heart within the ribcage;

FIG. 2 is an illustration of heat anatomy showing the location of the ventricles;

FIG. 3 is an illustration of electrode placement in a five electrode ECG configuration;

FIG. 4A is a graph showing a typical ECG pulse of a healthy heart;

FIG. 4B is a graph showing an ECG of a fibrillating heart;

FIG. 5 is an illustration of electrode placement in a five electrode ECG configuration including a multi-electrode pad;

FIG. 6A is an oblique view of a multi-electrode pad with position indicators on the top surface;

FIG. 6B shows an exploded view of the multi-electrode pad with segmented electrodes on a lower layer;

FIG. 7 is a block diagram of a ventricle location determination apparatus;

FIG. 8 is a flow diagram showing steps involved in ventricle location determination;

FIG. 9 is a schematic illustration of a chest compression apparatus;

FIG. 10 is an oblique view of a stretcher board; and

FIG. 11 is an oblique view of a patient on a stretcher board with a chest compression apparatus.

### DETAILED DESCRIPTION

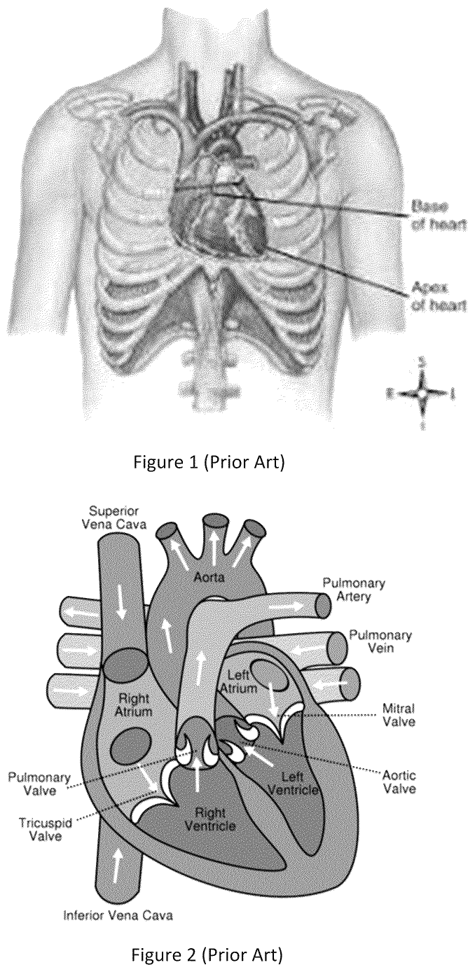

As shown in FIG. 1, the apex of the heart is positioned at the bottom left while the atria are located above and to the right. As illustrated in FIG. 2, the apex portion of the heart comprises the right and left ventricles, the right ventricle pumping blood to the lungs, and the left ventricle pumping blood to the rest of the body.

During CPR, a patient's heart is no longer pumping well enough, and the patient typically loses consciousness. A chest compression over the ventricles can be used to push blood out of ventricles into the pulmonary artery and aorta. Relaxation of the compression can allow blood to refill the ventricles from the atria. The rate of chest compressions may be roughly two per second, and roughly every twelve compression air is pushed into the lungs. The goal is to maintain sufficient oxygenated blood flow to the brain until such time as the heart may restart.

As mentioned above, if the location of chest compressions is not directed to the region of the ventricles, but instead over the atria or to a side of the ventricles, the flow of blood due to the chest compressions may be insufficient. While FIG. 1 shows a typical or average location of the heart within the chest cavity, there is sufficient variation of this location from person to person to frequently adversely affect CPR efficacy. While a blood flow or blood pressure measurement or a blood oxygen level measurement can inform the person performing CPR whether CPR is working or failing, it can be a challenge to determine how to make adjustments to the location of chest compressions when performing CPR in the case that it is failing.

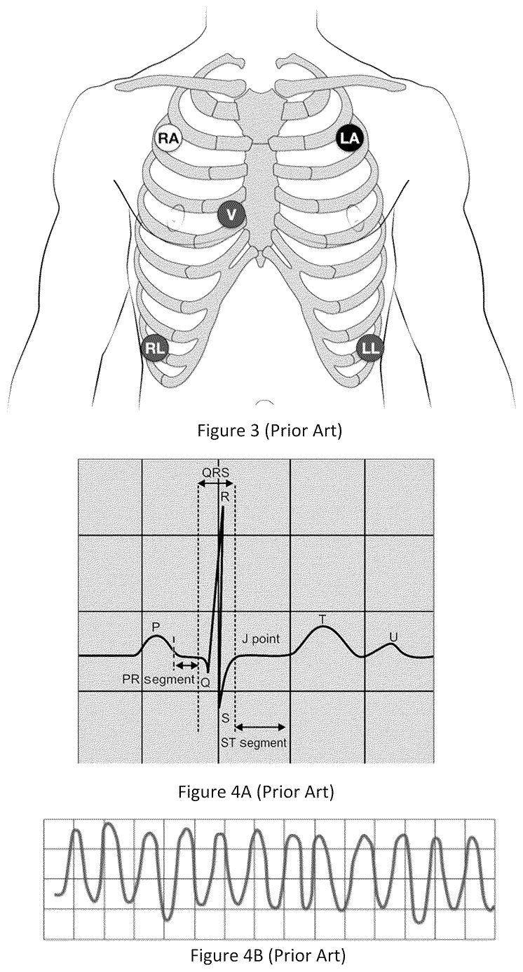

FIG. 3 illustrates an example of placement of electrocardiogram (ECG) electrode placement on a patient in the case of five electrodes. ECG may be performed with more or fewer electrodes and their placement on the body varies in accordance with the chosen ECG technique.

FIG. 4A illustrates an ECG signal from a single beat of a healthy heart, as is known in the art. For the purposes of determining the position of the ventricles of a patient's heart, Applicant proposes to use the signal strength or amplitude of the ECG signal as electrodes are at different locations on the body. For example, the V electrode may be at different positions over the heart region.

One of the most common cardiac rhythms during cardiac arrest is ventricular fibrillation. This rhythm is characterized by rapid, chaotic, and uncoordinated depolarization of myocardial cells in the ventricles. The rate is about 150 to 300 per minute and is illustrated in FIG. 4B. Because of this, usual ECG waves (P-Q-R-S-T) as illustrated in FIG. 4A cannot be identified. Since the electrical activity originates from the ventricles and the biggest myocardial mass is in the left ventricles, it is the applicants'belief that the analysis of the electrical signals can help identify the left ventricle position non-invasively and allow optimal chest compression position early in cardiac arrest.

In sinus rhythm, the amplitude of the ECG signal (more precisely of the QRS waves) is associated with the electrodes position in relation to the heart. (Medical & Biological Engineering & Computing, Volume 52, P109-119, 2014). This may be explained by the proximity to the heart ventricles and the ventricles'mass (and thus the electrical potential).

To identify the optimal location, the applicants propose to use two different analyses. In the first analysis, the total amplitude (absolute maximum value+absolute minimum value) is calculated over multiple second window (e.g., 10 seconds). The electrode position with the highest amplitude is then identified as the optimal chest compression site.

In the second analysis, the amplitude spectrum area (the sum of products of frequencies and amplitude) of the ventricular fibrillation signal, over a multiple-second window (e.g. 10 seconds), is calculated for each electrode and the electrode with the highest amplitude spectrum area (AMSA) value is identified as the optimal chest compression site. To calculate the AMSA, an FFT is performed on the signal segments after a windowing function, such as a Hamming window, is applied to reduce spectral leakage. The frequency range of interest (for example 0.5-40 Hz) is then extracted based on the sampling frequency and FFT resolution. The amplitude spectrum is computed as the magnitude of the FFT output for each frequency bin within this range. Finally, the AMSA is obtained by summing the amplitude spectrum values, multiplied by the frequency resolution. These analyses can be performed independently or simultaneously and repeatedly over time.

While using both of these two analyses is more reliable than using only one to determine the best location for the location of the ventricles, it will be appreciated that a single analysis may suffice.

Another common cardiac rhythm during cardiac arrest is pulseless electrical activity (PEA). This rhythm is characterized by a sinusoidal electrical activity without mechanical activity. Because of this, usual ECG waves can be identified. Since the electrical activity originates from the ventricles and that the biggest myocardial mass is in the left ventricles, it is the applicants'belief that the analysis of the electrical signals can help identify the left ventricle position non-invasively and allow optimal chest compression position early in cardiac arrest. These analyses can be performed simultaneously and repeatedly over time.

In this case, to identify the optimal location, the applicants propose to use two different analyses. The total amplitude (absolute maximum value+absolute minimum value is calculated over multiple second window (e.g. 10 seconds). The electrode with the highest amplitude is then identified as the optimal chest compression site. In the second analysis R-wave amplitude is measured for every electrical cycle. A mean R-Wave value is calculated over a multiple second window (e.g. 10 seconds). These analyses can be performed independently or simultaneously and repeatedly over time. The R-Wave is selected because its value is maximal over the left ventricle, whereas the Q-Wave is maximal over the right ventricle (Medical & Biological Engineering & Computing, Volume 52, P109-119, 2014).

While multiple physiological and anatomical factors influence the QRS amplitude, the proximity of the ECG electrode to the heart directly impacts the QRS amplitude (the closer the electrode to the heart, the bigger the QRS amplitude). On an individual patient, the lead with the highest amplitude should represent the closest to the ventricle. By repeating this analysis in multiple axis and with both antero-posterior and sterno-apical pads this would allow us to identify the closest electrode to the ventricle. This site may be chosen to perform chest compression to optimize left and right ventricular compression fraction and to minimize chances of left ventricular outflow track compression since this structure is in the upper part of the heart near the atria. This electrode may be identified by a visual clue (e.g., LED) to advise the resuscitator on the optimal chest compression site. The optimal compression site may also be transferred to the central algorithm to place the active compression/decompression pad at the optimal location.

While the apparatus described herein are for determining the position of the ventricles in a patient having heart failure, it will be appreciated that in a patient at risk of heart failure or heart attack, it is possible to pre-emptively mark a patient or identify in the patient the location of the ventricles for receiving cardiopulmonary resuscitation (CPR) in the future. The method can involve collecting ECG signals from the patient with at least one electrode placed over a heart region on a chest wall at a plurality of positions. In the case of a functioning heart, the amplitude of the ECG signals can be taken from the QRS amplitude. This can be done with a plurality of electrodes 20, as for example with the multi-electrode pad, or by using a single moveable electrode 20. Then one can determine from an amplitude of the ECG signals a location of the ventricles of the patient. An indication of the location can then be provided to a person for performing CPR on the patient. This indication can be stored in a database or an electronically readable medical bracelet and retrieved by EMT personnel. It can provide an image of the patient's chest showing the location, or measurements of the location with respect to an anatomical reference such as the ribs or sternum. Alternatively, marking a skin of the patient with the location, for example using an indelible dye marker or tattoo can be used.

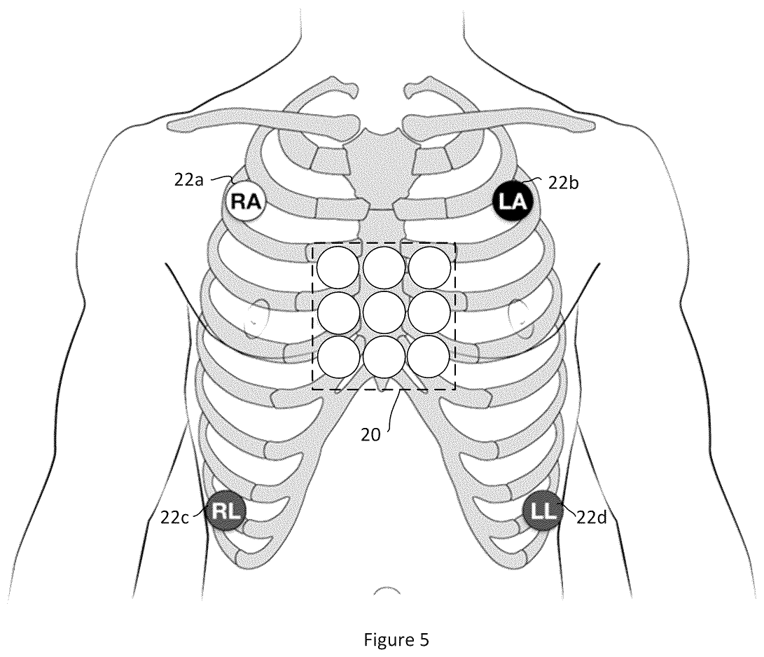

While it is possible to move a single electrode 20 over the heart region to determine where on the patient's chest the strongest ECG amplitude is found, this is not always suitable when compressions need to start in the absence of a strong enough ECG signal for ventricle locating purposes. As illustrated in FIG. 5, it is preferred to provide an arrangement of electrodes 20, such as a matrix, with the goal of obtaining ECG signals from a variety of positions on the body so that such signals can be obtained during the application of chest compressions.

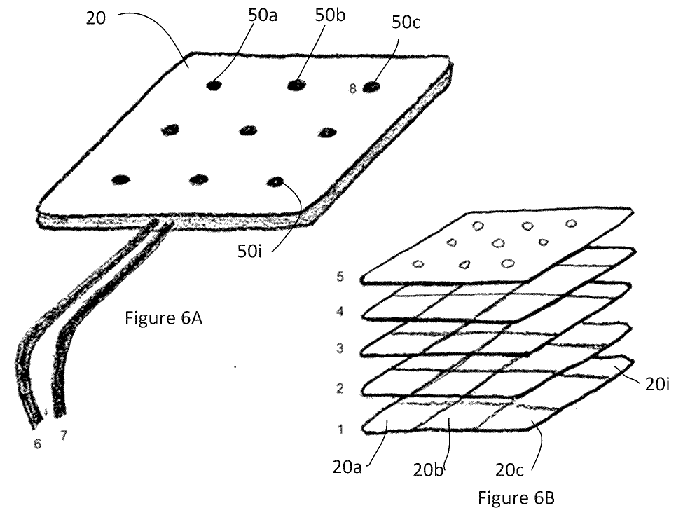

FIG. 6A provides an example of a multi-electrode pad 20 that is to be adhered to the chest with a suitable conductive adhesive. Suitable conductive adhesive is known in the art of ECG electrodes. However, the large surface area pad 20 on the skin side is broken up into 9 electrodes (any number greater than 3 may be practical) 20a through 20i.

An exploded view of a combined ECG monitoring and defibrillator pad is presented in FIG. 6B. In one proposed embodiment of the invention, the defibrillator pad may be composed of multiple layers: a stimulating conductive polymer layer 1, a monitoring polymer layer 2, a conductive metal layer 3, an insulating foam cover layer 4 and a LED layer 5. Pad 20 may be connected to a defibrillator/monitor using a stimulating wire 6 and a monitoring wire 7. The pad 20 may also contain multiple LEDS 8.

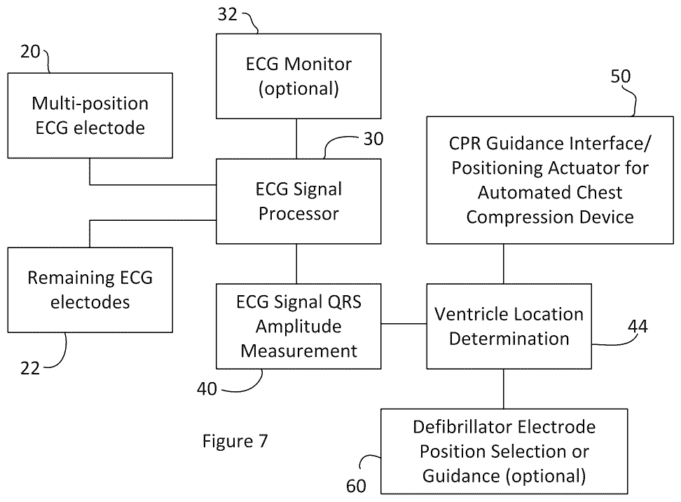

FIG. 7 is a block diagram of a proposed apparatus for determining ventricular position based on ECG signals. An ECG signal processor 30 is connected to ECG electrodes, for example a multi-position electrode 20 and the remaining ECG electrodes 22. A monitor 32 may be provided to display the ECG, for example the best quality or strongest amplitude ECG. The ECG signal obtained using one multi-position electrode 20 and the remaining ECG electrodes 22 from processor 30 is then analyzed by the amplitude measurement unit 40 to extract the desired amplitude parameter.

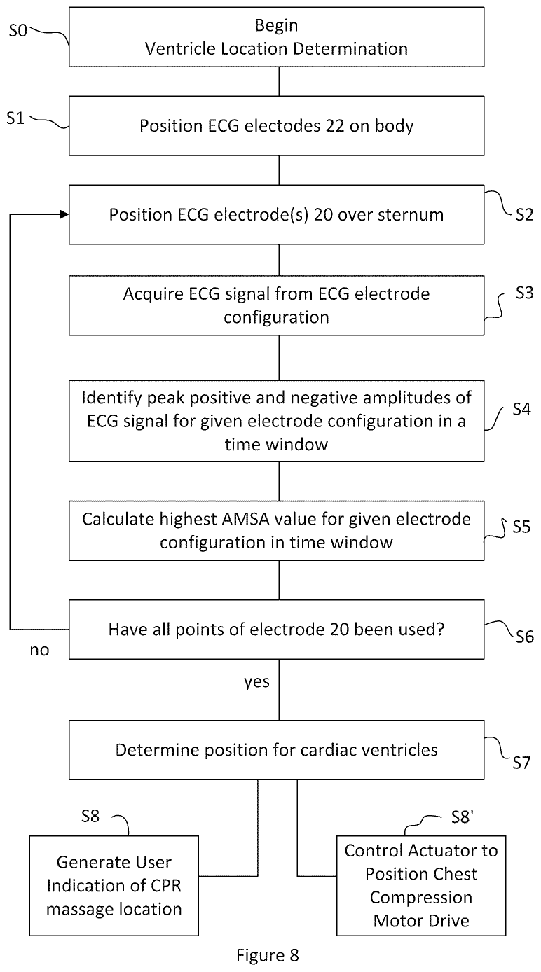

The ventricle location determination module 44 receives the amplitudes for the ECG measurements using the multi-position electrode 20 and calculates the ventricle position. The ventricle position or location determination process will be described below with reference to FIG. 8. For performing CPR, the ventricle position information is either provided to an interface unit for guiding a person performing CPR or to a positioning actuator unit 50. Knowledge of the position of the heart may alternatively be used for selecting defibrillator electrode locations, for example using unit 60 for defibrillator electrode position selection or guidance. Thus, a defibrillator device can direct a defibrillation charge to electrodes that are positioned on the patient so as to have the best result on the heart.

It will be appreciated that the integration of ventricle location determination functionality into a conventional AED can improve the outcome of patients receiving CPR and being treated by an AED device. It will be appreciated that an AED device only helps patients whose hearts are in a state of shockable rhythm. In such a state, the heart is failing to provide adequate blood circulation, and the patient will lose consciousness. When an AED device is connected to a patient in cardiac arrest, the AED device may alert that the patient in not in a state of shockable rhythm, and that CPR is required. When CPR is performed, the heartbeat may return either as non-shockable or as shockable. When the returned heartbeat is shockable, the AED device may warn the person performing CPR to stop so that a defibrillation pulse may be discharged to the patient. Defibrillation serves to “reset” a heartbeat that is non-perfusing to become a regular heartbeat and is not used to start an asystolic heart.

Therefore, by modifying at least one of the defibrillator electrodes of an AED to be a multi-electrode for ECG monitoring purposes, the AED device may not only alert that CPR is required, but also be adapted to determine the ventricle position with respect to the multi-electrode pad. By giving the instruction to apply chest compressions at a location referenced on the pad as determined from the ECG signals, the effectiveness of the CPR is more likely.

The steps involved in ventricle position or location determination are illustrated according to one embodiment in FIG. 8. At step S0, the process begins. At step S1, the body electrodes 22 are attached to the skin of the patient in accordance with the chosen ECG methodology. In the case of a multi-electrode array 20, for example, one placed on the lower sternum area, step S2 involves placing the pad 20 on the patient. The processor 30 then collects the signals from electrodes 20 and 22 in step S3. Unit 40 then analyzes the ECG signals to identify the absolute maximum and minimum value of the ECG signal within a given time window, typically of at least a second and more typically around 10 seconds, at step S4. Step S5 the AMSA is calculated on the ECG signal in a time window that may the same or different from the time window in S4. While it is preferable to measure and record the ECG signals from all positions of the multi-electrode 20 simultaneously, it is also possible to do so sequentially, for example over different pulses and with averaging. In this case, at step S6, one redirects the method to step S2 until all electrode positions of electrode 20 are measured and with sufficient averaging.

In step S7, the electrical potential source location in the heart is determined. This may be done by taking the greatest value from steps S4 and S5 and the corresponding electrode 20 position. Alternatively, an interpolation may be done if the electrode 20 spacing is great enough.

In step S8, the position of the cardiac ventricles is output to the user. This may take the form of selectively illuminating a small LED 50a through 50i on a top surface of pad 20. This may also take the form of a spoken word audio output to provide the location with reference to indicia provided on pad 20. AED devices are known to use spoken word audio output to provide instructions and information to the user of the device.

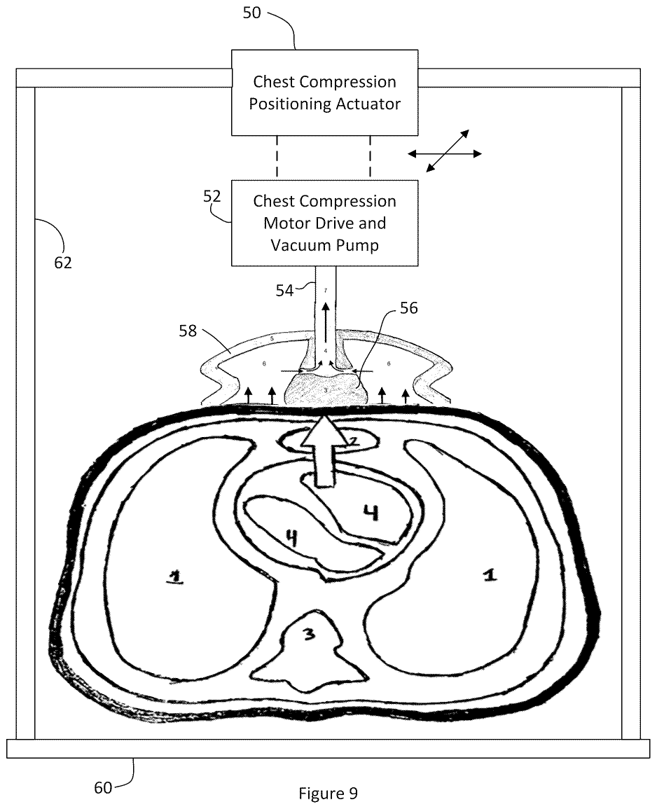

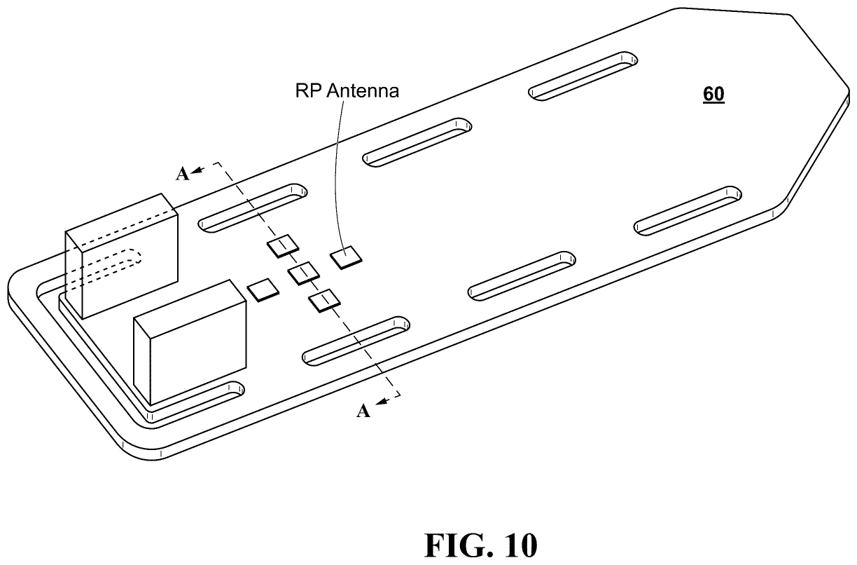

In step S8′, the position of the cardiac ventricles is output to an actuator to position the mechanical chest compression device accordingly. As shown in FIG. 9, a panel 60 may be placed under the back of the patient, and a frame 62 can be attached to the panel 60 to support the positioning actuator 50 and the chest compression motor drive 52. In this case, the frame 62 may have a guide, such as a laser guide, for ensuring that the technician places the pad 20 at a known location with respect to the actuator 50. In FIG. 9, a negative pressure flexible cover 58 is connected via a conduit 54 to a vacuum or suction source in the motor drive 52 so that lifting of the shaft 54 and the chest compression palm 56 helps to pull on the chest to expand the ribcage using the cover 58. In this way, the mechanical chest compression device 52 can perform ventricle compression and ribcage expansion for better circulation of oxygenated blood.

If the determined position of the cardiac ventricles based on the ECG signals were to change after the onset of chest compressions, the vacuum source can be interrupted briefly to allow the palm 56 and cover 58 to be lifted from the chest so that the actuator 50 can reposition the palm 56 to be more accurately over the expected location of the ventricles. Then the vacuum source can resume along with the regular chest compressions and ventilation.

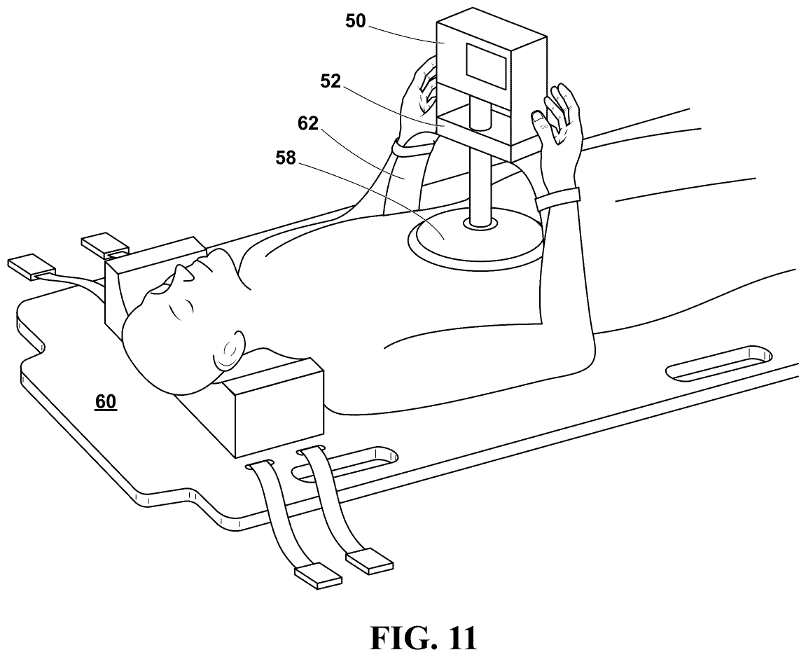

FIG. 10 illustrates a patient stretcher board that includes panel 60. FIG. 11 shows schematically a patient positioned on the stretcher board with the automated chest compression device mounted to the panel 60.

It is explicitly stated that all features disclosed in the description and/or the claims are intended to be disclosed separately and independently from each other for the purpose of original disclosure as well as for the purpose of restricting the claimed invention independent of the composition of the features in the embodiments and/or the claims. It is explicitly stated that all value ranges or indications of groups of entities disclose every possible intermediate value or intermediate entity for the purpose of original disclosure as well as for the purpose of restricting the claimed invention, in particular as limits of value ranges.

1. An apparatus for determining a location of a heart on a patient, the apparatus comprising:
a pad carrying an array of ECG electrodes to be placed over an area of the heart on a patient's chest;
a plurality of body electrodes;
an ECG device connectable to said pad and said plurality of body electrodes for generating an ECG signal for each electrode of said array of ECG electrodes; and
a signal analyzer connectable to said ECG device for determining a location with respect to said array of ECG electrodes corresponding to a location of heart ventricles.

2. The apparatus of claim 1, wherein the signal analyzer is configured to measure a maximum and a minimum of an amplitude of said ECG signal for each electrode of said array of ECG electrodes over a given time window.

3. The apparatus of claim 2, wherein the signal analyzer is configured to measure an amplitude spectrum area (AMSA) an amplitude of said ECG signal for each electrode of said array of ECG electrodes over a given time window.

4. The apparatus of claim 1, wherein the signal analyzer is configured to measure an amplitude spectrum area (AMSA) an amplitude of said ECG signal for each electrode of said array of ECG electrodes over a given time window.

5. The apparatus of claim 1, further comprising an indicator connected to the signal analyzer for providing an indication of said location of heart ventricles.

6. The apparatus of claim 5, wherein said indicator comprises LEDs located on said pad.

7. The apparatus of claim 5, wherein said indicator comprises a CPR guidance interface.

8. The apparatus of claim 7, wherein said interface comprises an audio speaker.

9. An automated cardiopulmonary resuscitation (CPR) system comprising:
an apparatus for determining a location of a heart on a patient as defined in claim 1;
a chest compression motor drive having a chest compression pad; and
a positioning actuator for positioning said chest compression motor drive, said positioning actuator connected to said signal analyzer.

10. The system of claim 9, wherein the signal analyzer is configured to measure a maximum and a minimum of an amplitude of said ECG signal for each electrode of said array of ECG electrodes over a given time window.

11. The system of claim 10, wherein the signal analyzer is configured to measure an amplitude spectrum area (AMSA) an amplitude of said ECG signal for each electrode of said array of ECG electrodes over a given time window.

12. The system of claim 9, wherein the signal analyzer is configured to measure an amplitude spectrum area (AMSA) an amplitude of said ECG signal for each electrode of said array of ECG electrodes over a given time window.

13-14. (canceled)

15. An automated external defibrillator (AED) system comprising:
an apparatus for determining a location of a heart on a patient as defined in claim 1;
two defibrillation electrodes;
an interface connected to said signal analyzer for providing an indication of said location of heart ventricles and at least warning of defibrillation; and
a defibrillator connected to said ECG device, said interface and to said two defibrillation electrodes for determining when a defibrillation charge should be automatically administered, providing through said interface said warning of defibrillation and delivering said defibrillation charge.

16. The system of claim 15, wherein the signal analyzer is configured to measure a maximum and a minimum of an amplitude of said ECG signal for each electrode of said array of ECG electrodes over a given time window.

17. The apparatus of claim 16, wherein the signal analyzer is configured to measure an amplitude spectrum area (AMSA) an amplitude of said ECG signal for each electrode of said array of ECG electrodes over a given time window.

18. The apparatus of claim 15, wherein the signal analyzer is configured to measure an amplitude spectrum area (AMSA) an amplitude of said ECG signal for each electrode of said array of ECG electrodes over a given time window.

19. The apparatus of claim 15, wherein said interface comprises LEDs located on said pad.

20. (canceled)

21. A cardiopulmonary resuscitation (CPR) system comprising:
a pad carrying an array of ECG electrodes to be placed over an area of the heart on a patient's chest;
a plurality of body electrodes;
an ECG device connectable to said pad and said plurality of body electrodes for generating an ECG signal for each electrode of said array of ECG electrodes;
a signal analyzer connectable to said ECG device for determining a location with respect to said array of ECG electrodes corresponding to a location of heart ventricles;
a chest compression motor drive having a chest compression pad connected to a vacuum pump;
a frame connectable to a panel to be placed under a back of a patient, said frame supporting said chest compression motor drive; and
one of:
a CPR guidance interface controlled by the signal analyzer to indicate the location of the heart ventricles to guide a technician to position the frame to have the chest compression pad positioned over the heart ventricles; or
a positioning actuator mounted to the frame, wherein the pad is at a known location with respect to the frame and the positioning actuator is connected to said signal analyzer to position the chest compression pad at the location of the heart ventricles.

22. The system as defined in claim 21, wherein said CPR guidance interface comprises indicators on a top surface of the pad.

23. The system as defined in claim 21, wherein the frame comprises a laser guide for assisting a technician to place the pad at a known location with respect to the frame so that with the pad at the known location, the positioning actuator can position the chest compression pad at the location of the heart ventricles.
